(19) United States
(12) Patent Application Publication
(10) Pub. No.: US 2018/0263992 A1
(43) Pub. Date: Sep. 20, 2018

(54) TREATMENT OF NEURODEGENERATIVE DISEASES

(71) Applicant: BENEVOLENTAI BIO LIMITED, London, Greater London (GB)

(72) Inventors: Peter RICHARDSON, London, Greater London (GB); Richard MEAD, Sheffield, South Yorkshire (GB); Laura FERRAIUOLO, Sheffield, South Yorkshire (GB); Kenneth Patrick MULVANY, London (GB)

(21) Appl. No.: 15/762,657

(22) PCT Filed: Sep. 23, 2016
(86) PCT No.: PCT/GB2016/052970

§ 371 Date: Mar. 23, 2018

(30) Foreign Application Priority Data
Sep. 24, 2015 (GB) 1516905.5

(51) Int. Cl. A61K 31/5377; A61K 31/49; A61K 31/4745; A61K 31/473

(52) U.S. Cl. CPC A61K 31/5377; A61K 31/473; A61K 31/4745; A61K 31/49

(57) ABSTRACT

Methods for the prevention and treatment of neurodegenerative diseases, in particular motor neuron diseases such as amyotrophic lateral sclerosis (ALS), is described, as well as compositions and combined preparations for use in the methods. The methods comprise inhibiting EGFR signalling, and inhibiting MyD88-dependent TLR/IL-R1 signalling, in the central nervous system of a subject in need of such prevention or treatment. The compositions comprise an inhibitor of EGFR signalling, and an inhibitor of MyD88-dependent TLR/IL-R1 signalling.

This invention relates to the treatment of neurodegenerative diseases, in particular motor neuron diseases such as amyotrophic lateral sclerosis, and to compositions or combined preparations for use in the methods.

The motor neuron diseases (MNDs) are a group of progressive neurological disorders that destroy motor neurons, the cells that control essential voluntary muscle activity such as speaking, walking, breathing, and swallowing. Normally, messages from nerve cells in the brain (upper motor neurons) are transmitted to nerve cells in the brain stem and spinal cord (lower motor neurons) and from them to particular muscles. Upper motor neurons direct the lower motor neurons to produce movements such as walking or chewing. Lower motor neurons control movement in the arms, legs, chest, face, throat, and tongue.

When there are disruptions in the signals between the lowest motor neurons and the muscle, the muscles do not work properly; the muscles gradually weaken and may begin to waste away and develop uncontrollable twitching (called fasciculations). When there are disruptions in the signals between the upper motor neurons and the lower motor neurons, the limb muscles develop stiffness (called spasticity), movements become slow and effortful, and tendon reflexes such as knee and ankle jerks become overactive. Over time, the ability to control voluntary movement can be lost.

MNDs are classified according to whether they are inherited (familial) or sporadic, and to whether degeneration affects upper motor neurons, lower motor neurons, or both. In adults, the most common MND is amyotrophic lateral sclerosis (ALS), which affects both upper and lower motor neurons. It has inherited and sporadic forms and can affect the arms, legs, or facial muscles. Primary lateral sclerosis (PLS) is a disease of the upper motor neurons, while progressive muscular atrophy (PMA) affects only lower motor neurons in the spinal cord. In progressive bulbar palsy (PBP), the lowest motor neurons of the brain stem are most affected, causing slurred speech and difficulty chewing and swallowing. There are almost always mildly abnormal signs in the arms and legs.

TABLE 1
Classification of Motor Neuron Diseases

| | UMN | LMN |
|---|---|---|
| Type | degeneration | degeneration |
| Amyotrophic lateral sclerosis (ALS) | Yes | Yes |
| Primary lateral sclerosis (PLS) | Yes | No |
| Progressive muscular atrophy (PMA) | No | Yes |
| Progressive bulbar palsy (PBP) | No | Yes, |
| | | bulbar region |
| Pseudobulbar palsy | Yes, | No |
| | bulbar region | |

ALS is a progressive, ultimately fatal disorder that disrupts signals to all voluntary muscles. The terms motor neuron disease and ALS are often used interchangeably. ALS most commonly strikes people between 40 and 60 years of age, but younger and older individuals also can develop the disease. Men are affected more often than women. Familial forms of ALS account for 10 percent or less of cases of ALS, with more than 10 genes identified to date. However, most of the gene mutations discovered account for a very small number of cases. The most common familial forms of ALS in adults are caused by mutations of the superoxide dismutase gene, or SOD1, located on chromosome 21.

There is no cure or standard treatment for ALS or the other MNDs. Riluzole (Rilutek®), the only prescribed drug approved by the U.S. Food and Drug Administration to treat ALS, prolongs life by 2-3 months but does not relieve symptoms, and has undesirable side effects such as nausea and fatigue. The drug reduces the body's natural production of the neurotransmitter glutamate, which carries signals to the motor neurons. It is believed that too much glutamate can harm motor neurons and inhibit nerve signalling.

The mammalian central nervous system (CNS) is considered to be immunologically privileged, with relatively few resident immune cells and a highly specific blood-brain barrier (BBB). However, considerable evidence supports the presence of immune and inflammatory abnormalities in neurodegenerative diseases. Neuroinflammation is characterised by the activation and proliferation of microglia (microgliosis), astrogliosis, and infiltrating immune cells. It is a pathological characteristic of many neurodegenerative diseases including Alzheimer's disease (AD), Parkinson's disease (PD), and ALS. Neuroinflammatory responses can be beneficial or harmful to motor neuron survival. These distinct effects are elicited by the different activation states of microglia/macrophages and astrocytes, and are modulated by infiltrating T cells (Zhao et al., J Neuroimmune Pharmacol. 2013; 8(4): 888-899).

Microglia act as the first line of immune defence in the CNS, surveying the surrounding environment through their processes. Microglia are sensitive to pathological changes in the CNS and respond to danger signals from damaged tissue. During the early stage of motor neuron injury in ALS, it is believed that repair signals from motor neurons induce activation of microglial cells to an M2 phenotype. M2 microglia release neuroprotective factors (such as neurotrophic and anti-inflammatory factors) to repair motor neurons and protect against further injury. Astrocytes also participate in the neuroprotective process by secreting neurotrophic factors. As the disease progresses, injured motor neurons release danger signals that transform microglia to a cytotoxic M1 phenotype. M1 microglia release pro-inflammatory cytokines (such as tumor necrosis factor α, TNF-α, and interleukin-1β, IL-1β), and promote neurotoxicity by releasing reactive oxygen species. These pro-inflammatory cytokines further activate microglia leading to excessive neurotoxicity. M1 microglia also promote astrocyte activation. Activated astrocytes acquire deleterious inflammatory phenotypes with release of reactive oxygen species and pro-inflammatory cytokines, which in turn further induce microglial activation and enhance motor neuron degeneration. The activated glial cells also recruit peripheral monocytes/macrophages and T cells into the CNS, which further exacerbate motor neuron degeneration. The neuroinflammatory response in ALS is reviewed by Zhao et al., supra, and by Lewis et al. (Neurology Research International, 2012, Article ID 803701).

Pattern recognition receptors (PRRs) expressed primarily on microglia are the initial responders to tissue insult or damage. PRRs detect unique microbial structures termed pathogen-associated molecular patterns (PAMPs), for example microbial nucleic acids, bacterial secretion systems, and components of the microbial cell wall. Damaged host cells can also trigger PRRs by releasing danger-associated molecular patterns (DAMPs) such as uric acid crystals, ATP, high-mobility group box 1 (HMGB1), and the heat-shock proteins hsp70 and hsp90. PRRs may either be on the membrane surface, for example Toll-like receptors (TLRs) and C-type Lectin Receptors (CLRs), or inside the cytoplasm, for example Nod-like receptors (NLRs), RIG-I-like receptors (RLRs), and AIM2-like receptors (ALRs). Many PRRs encountering PAMPs and DAMPs trigger signalling cascades that promote gene transcription by nuclear factor-kB (NF-kB), activator protein 1 (AP1), and interferon regulatory factors (IRFs). Target genes encode cytokines, interferons, and other proinflammatory or microbicidal proteins.

The Toll-like receptor/Interleukin-1 receptor (TLR/IL-1R) superfamily is a group of structurally homologous proteins characterized by extracellular immunoglobulin-like domains and an intracellular Toll/Interleukin-1R (TIR) domain. The members of the TLR/IL-1R superfamily play a fundamental role in the immune response. These receptors detect microbial components and trigger complex signalling pathways that result in increased expression of multiple inflammatory genes. The superfamily includes the Toll-like receptor (TLR) subfamily, the Interleukin-1 receptor (IL-1R) subfamily, and TIR-domain-containing adaptor proteins (such as MyD88).

A subset of NLRs and ALRs triggers a distinct defence mechanism. These proteins assemble cytosolic protein complexes called inflammasomes. Once active, the inflammasome binds to pro-caspase-1 (the precursor molecule of caspase-1), either via its own caspase activation and recruitment domain (CARD), or via the CARD of the adaptor protein ASC, which it binds to during inflammasome formation. The inflammasome induces autocatalytic cleavage of pro-caspase-1 molecules to form caspase-1, which can carry out a variety of processes in response to the initial inflammatory signal, including the proteolytic cleavage of pro-interleukin (IL)-113 into IL-16, a pro-inflammatory cytokine.

IL-1β signals through the type I IL-1 receptor/IL-1 accessory protein (IL-1RAcP) complex, leading to NFkB-dependent transcription of pro-inflammatory cytokines (tumor necrosis factor (TNF)-α, IL-6, and interferons) and neutrophil-recruiting chemokines (CXCL1 and CXCL2) in glia. IL-1β induces expression of its own gene (by activating NF-κB), which serves as a positive feedback loop that amplifies the IL-1 response.

RNA-binding proteins, and in particular transactive response (TAR) DNA-binding protein 43 (TDP43), are central to the pathogenesis of motor neuron diseases and related neurodegenerative disorders. TDP43 is the major constituent of proteinaceous inclusions that are characteristic of most forms of ALS. TDP43 contains two RNA-recognition motifs (RRMs) involved in RNA and DNA binding, and a glycine-rich carboxy-terminal domain. TDP43 is predominantly nuclear localised. Pathological TDP-43 found in diseased brain and spinal cord is abnormally aggregated, primarily in the cytoplasm. Nearly all sporadic and TDP43 mutant familial cases have TDP43 aggregations (Lee et al., Nat Rev Neurosci. 2011 Nov. 30; 13(1):38-50). The precipitated TDP43 protein is polyphosphorylated, and ubiquitylated. Phosphorylation is tightly associated with aggregation. Acetylation of TDP43 is also part of the aggregation process. Acetylation impairs RNA-binding and promotes accumulation of insoluble, hyper-phosphorylated TDP43 species that largely resemble pathological inclusions in ALS. Acetylation occurs on lysine residues within the RRMs of TDP43. The cytoplasmic histone deacetylase 6 (HDAC6) interacts with TDP43 in viva HDAC6 has been shown to deacetylate TDP43, although cytoplasmic TDP43 aggregates were unable to be deacetylated efficiently (Cohen et al., Nat Commun. 2015 Jan. 5; 6:5845).

The SOD1^{G93A} mouse model of ALS is the most widely used animal model for ALS (Gurney et al., 1994, Science 264: 1772-1775). In these mice, a familial mutation in the human SOD1 gene (G93A) that causes ALS is expressed transgenically throughout the body under the control of the endogenous mouse SOD1 promoter. The transgene insertion causes a degenerative disease of lower motor neurons leading to progressive paralysis and eventual death, with the number of transgene copies correlating with severity of disease, Cytoplasmic mislocalization of TDP43 occurs at the end-stage of disease.

The mSOD mouse model recapitulates many aspects of the neuroinflammatory response observed in ALS patients. In the mSOD mouse, increased numbers of activated microglia are observed at early pre-symptomatic stages of disease, and with disease progression to end-stage, microglial numbers in the lumbar spinal cord increase further by nearly 2-fold. Several studies have demonstrated that modulation of the inflammatory response in mSOD mice alters disease progression, leading to suggestions that microgliosis in the mSOD mouse contributed to motoneuron degeneration. However, experiments in which the proinflammatory cytokine TNF-α was ablated in mSOD mice, or where the proliferation of microglia was blocked, had no effect on the rate of disease progression, suggesting that microgliosis does not exacerbate neurodegeneration in the mSOD mouse model.

Some evidence suggests that the epidermal growth factor receptor (EGFR) signalling pathway could play a role in the pathology of neurodegenerative conditions. Treatment with EGFR inhibitors is reportedly neuroprotective in both a rat model of glaucoma (Liu et al., 2006, J Neurosci 26: 7532-7540) and a rat model of spinal cord injury (Erschbamer et al., 2007, J Neurosci 27: 6428-6435). In both studies the authors suggest that EGFR inhibition targets reactive astrocytes. Furthermore, EGFR mRNA expression was found to be upregulated over 10-fold in the spinal cord of human ALS patients as well as in that of the SOD1^{G93A} mouse model (Offen et al., 2009, J Mol Neurosci 38: 85-93), suggesting that pharmacological inhibition of EGFR signalling could be a feasible strategy to slow progression of this disease.

EGFR levels in the spinal cord of SOD1 mice and patients with ALS are increased some 10-fold compared to controls (Offen et al., J Mol Neurosci. 2009 June; 38(2):85-93). The EGFR is strongly implicated in astrocyte activation as a consequence of spinal cord injury (Li et al., Neurochem Int. 2011 June; 58(7):812-9; Li et al., Journal of Neuroinflammation 2014, 11:71). Activated astrocytes express glial fibrillary acid protein (GFAP), inhibit axonal and dendrite elaboration, and release a variety of inflammatory cytokines including TNF, IL-1β and IL-6, which are able to induce neuronal apoptosis (Monje et al., Science. 2003 Dec. 5; 302(5651):1760-5). In addition EGFR inhibitors have been shown to promote neuronal regeneration, while EGF itself can increase astrocyte genesis (Kuhn et al., J Neurosci. 1997 Aug. 1; 17(15):5820-9). Thus, the EGFR can play a pivotal role in astrocyte activation in the spinal cord.

The EGFR is also involved in microglial activation and proliferation (Qu et al., J Neuroinflammation. 2012 Jul. 23; 9:178), with blockade of the EGFR dramatically reducing the microglial response to LPS. Similarly in vivo, EGFR blockade reduces microglial and astrocyte activation, scar formation and enhanced axonal outgrowth (Qu et at, 2012, supra).

These observations suggest that transformation of glial cells in ALS to a MN-toxic phenotype could be significantly modified by blockade of the EGFR. Le Pichon et al., 2013 (PLoS ONE, 8(4): e62342; 1-12) describe a study to test whether erlotinib, an EGFR inhibitor marketed for the treatment of non-small cell lung carcinoma, had a beneficial effect in the SOD1^{G93A} mouse model of ALS. The authors report that erlotinib penetrated into the central nervous system and resulted in a modest yet statistically significant symptom delay as measured by multiple readouts of disease onset and progression. However, the treatment failed to extend lifespan, did not protect motor synapses, and did not correlate with a modulation of markers for astrocytes and microglia. The authors conclude that erlotinib is not efficacious in treating the SOD1 mouse model of ALS. Given the lack of efficacy of erlotinib in this mouse model and the drug's undesirable side effects, which include skin irritation and diarrhea, the authors conclude that erlotinib does not appear to be a good clinical candidate for the treatment of ALS.

There remains, therefore, an urgent need for improved treatment of ALS, and other neurodegenerative diseases.

We have appreciated that effective treatment of neurodegenerative diseases, such as ALS, requires simultaneous correction of multiple dysfunctional pathways and processes.

According to the invention there is provided a method of preventing or treating a neurodegenerative disease, which comprises: inhibiting EGFR signalling; and inhibiting MyD88-dependent TLR/IL-R1 signalling; in the central nervous system (CNS) of a subject in need of such prevention or treatment.

There is also provided according to the invention an inhibitor of EGFR signalling, and an inhibitor of MyD88-dependent TLR/IL-R1 signalling, for use in the prevention or treatment of a neurodegenerative disease.

The invention also provides use of an inhibitor of EGFR signalling, and an inhibitor of MyD88-dependent TLR/IL-R1 signalling, in the manufacture of a medicament for the prevention or treatment of a neurodegenerative disease.

Within the CNS, EGFR signalling and/or MyD88-dependent TLR/IL-R1 signalling may be inhibited in microglia, astrocytes, or neurons, in microglia and astrocytes, in microglia and neurons, in astrocytes and neurons, or in microglia, astrocytes, and neurons.

The EGFR (also known as ErbB1 or HER1) is a member of the ErbB family of receptors. The other members of the family are ErbB2/HER2/Neu, ErbB3/HER3 and ErbB4/HER4. They are all transmembrane glycoproteins consisting of: (i) a cysteine-rich, extracellular N-terminal ligand binding domain and a dimerization arm; (ii) a hydrophobic transmembrane domain; and (iii) an intracellular, highly conserved, cytoplasmic C-terminal tyrosine kinase domain with several phosphorylation sites. The ectodomain of EGFR has a closed, inactive conformation, and an open, active conformation, which remain in equilibrium with each other. The closed conformation is favoured in the absence of a ligand. Binding of a ligand shifts the equilibrium and stabilizes the open conformation, allowing the dimerization arm to interact with an identical dimerization arm of another receptor molecule to form a homodimer. EGFR also promotes heterodimerization with other members of the HER family, including HER2, HER3 and HER4. Thus, EGFR can initiate cellular signalling cascades by itself, through homodimerzation, or through heterodimerization with other HER family members.

ErbB family members can be activated by 13 known ligands, including EGF, transforming growth factor alpha (TGF-α), amphiregulin (AR), betacellulin (BTC), heparin-binding EGF-like growth factor (HB-EGF), epiregulin (EPR), epigen (EPG) and neuregulins 1-6 (NRG). EGF, TGF-α, AR, BTC and EPR bind specifically to EGFR, Various ligands can induce specific heterodimerization, for example EGF can induce heterodimerization of EGFR with HER2, HER3 or HER4. Thus homo- and heterodimerization of EGF receptors facilitates complex signalling cascades (Seshacharyulu et al., Expert Opin Ther Targets, 2012 (16(1):15-31).

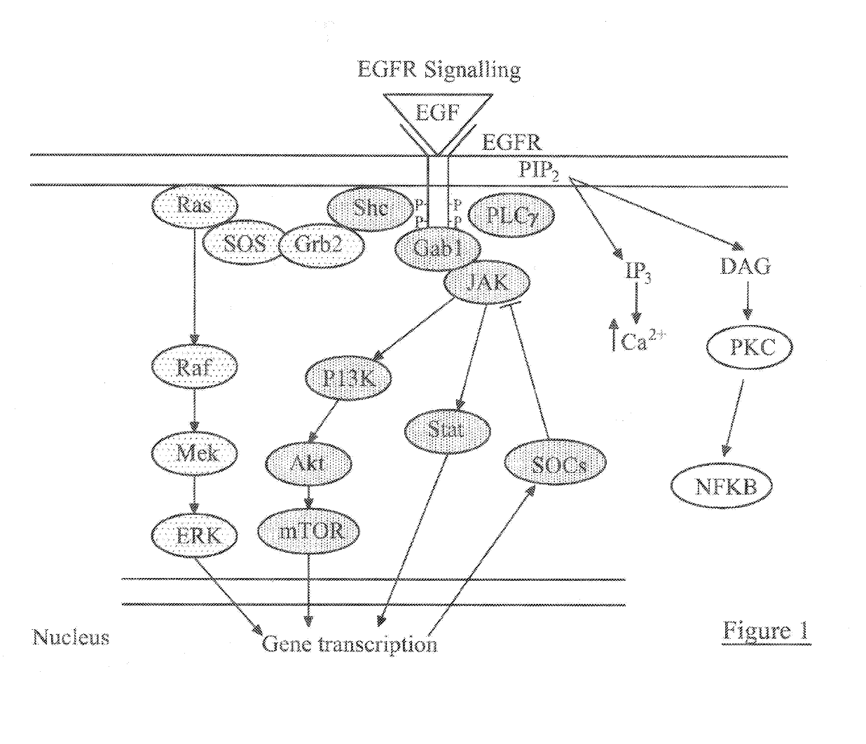

Activation of EGFR signalling (illustrated in FIG. 1) is triggered by ligand-induced receptor dimerization, following which the tyrosine residues present in the intrinsic kinase domain of one receptor cross phosphorylates specific residues (including Y992, Y1045, Y1068, Y1148, Y1173) in the C-terminal tail of the partnering receptor. Recruitment of effector proteins occurs via Src homology 2 (SH2) and phosphotyrosine binding (PTB) domains on the effector proteins, and the phosphotyrosine motif present on the intracellular tyrosine kinase domain of the receptor. On subsequent dissociation, activated adaptor and effector proteins further stimulate their corresponding signalling cascades, which include the KRAS-BRAF-MEK-ERK pathway, phosphoinositide 3-kinase (PI3K), phospholipase C gamma protein pathway, the anti-apoptotic AKT kinase pathway and the STAT signalling pathway. This leads to cell proliferation, angiogenesis, migration, survival, and adhesion.

One of the adaptor proteins, GRB2, binds to the phosphotyrosine residue at 1068 and recruits SOS to the membrane. SOS activates GDP/GTP exchange, which recruits RAF to the membrane. RAF phosphorylates MEKs, which then activates the extracellular signal regulated kinase (ERK). ERK activates a number of transcriptional regulators to induce cell growth and proliferation. GRB2 (or other adaptor proteins such as GABs) recruits PI3Ks, another major mediator of EGFR signalling. PI3Ks convert Phosphatidylinositol-4,5-bisphosphate (PIP2) to Phosphatidylinositol-3,4,5-trisphosphate (PIP3). PIP3 binds to PH domain of AKT and recruits it to plasma membrane. PDK1 phosphorylates AKT, which in turn regulates the activity of various proteins that mediate cell survival,

EGFR also activates phospholipase C, which hydrolyses PIP2 to generate Inositol trisphosphate (IP3) and 1,2-Diacylglycerol (DAG). IP3 induces the release of Ca²⁺ from endoplasmic reticulum to activate calcium-regulated pathways. DAG activates the protein kinase C pathway. One of the signalling modules regulated by PKC in the EGFR pathway is the NFκB module. The protein SRC is a key player in the activation of various pathways such as RAS, PLC and also the STAT proteins in various cells. Other signalling modules activated by EGFR include the FAK, JNK, p38MAPK and ERK5 modules. EGFR induces the JNK pathway through the activation of G proteins such as RAC and CDC42, which recruits JNK kinases as well as regulate the actin polymerization.

EGFR also translocates from the plasma membrane to other cellular compartments including the nucleus where it directly regulates the expression of several genes in cooperation with other transcriptional regulators such as STATs, PCNA and the E2F family of proteins.

In some embodiments, EGFR signalling may be inhibited by inhibiting tyrosine kinase activity of the EGFR, or at a point of the EGFR signalling pathway that is upstream of EGFR tyrosine kinase activity, for example by inhibiting binding of ligand to the receptor.

In view of the functional involvement of EGFR in various cellular processes, several approaches have been developed that target and interfere with EGFR-mediated effects. Two distinct therapeutic approaches currently employed for targeting EGFR in various human malignancies are the use of small molecule tyrosine kinase inhibitors, and monoclonal antibodies. Tyrosine kinase inhibitors target the intracellular tyrosine kinase domain of EGFR, whereas anti-EGFR antibodies bind to the extracellular domain of EGFR. Such inhibitors of EGFR signalling may be used in methods of the invention for the prevention or treatment of neurodegenerative diseases, especially motor neuron diseases.

Known tyrosine kinase inhibitors (TKO) of EGFR signalling are adenosine triphosphate (ATP) analogues. They inhibit EGFR signalling by competing and binding with ATP binding pockets on the intracellular catalytic kinase domain of receptor tyrosine kinases, thereby preventing auto-phosphorylation and activation of several downstream signalling pathways. Type I and II reversible inhibitors compete with ATP molecules that recognize the kinase active conformation. Irreversible inhibitors bind to the kinase active site covalently by specifically reacting with a nucleophilic cysteine residue. Irreversible inhibitors have the advantage of prolonged clinical effects and a decreased need for frequent dosing.

Examples of small molecule tyrosine kinase inhibitors that inhibit EGFR signalling include gefitinib, erlotinib, brigatinib, lapatinib, afatinib, and icotinib.

Gefitinib (ZD1839; Trade name Iressa) (N-(3-chloro-4-fluoro-phenyl)-7-methoxy-6-(3-morpholin-4-ylpropoxy)quinazolin-4-amine, described in U.S. Pat. No. 5,770,599) is approved for the treatment of patients with NSCLC after failure of both platinum-based or docetaxel chemotherapies. Gefitinib is an anilinoquinazoline-derived EGFR tyrosine kinase inhibitor.

It is an orally active low-molecular-weight EGFR inhibitor with selective tyrosine kinase activity. It does not inhibit serine-threonine kinase activity. Gefitinib has a 200-fold greater affinity for EGFR relative to the other ErbB family members. The biological half-life of gefitinib is 28 hours, and peak plasma concentration after it is absorbed is 3-7 hours. Clinical studies with gefitinib have revealed that dosages of 250 or 500 mg daily is effective against advanced lung cancer. The maximal tolerated dosage, assessed in phase I trials, was 700 mg/day. A suitable dosage range of gefitinib for the treatment of a neurodegenerative disease, such as ALS, is 100-750 mg per day, for example 250 mg per day.

Erlotinib (OSI-774; Trade name Tarceva) is another FDA-approved low molecular weight molecule similar to gefitinib, available in the form of an orally potent and selectively reversible inhibitor of EGFR tyrosine kinase. Like gefitinib, erlotinib functions as an ATP analogue by competing with ATP binding pockets within the receptor tyrosine kinases. Studies in human cancer cells found that it inhibits EGF-dependent cell proliferation at nanomolar concentrations and blocks cell-cycle progression in the G1 phase. Erlotinib is currently approved in patients with relapsed NSCLC and for maintenance therapy in advanced NSCLC patients whose disease had not progressed after four cycles of platinum-based first-line chemotherapy.

Lapatinib (GW-572016) is an orally active, reversible and specific receptor tyrosine kinase inhibitor of both EGFR and HER2. Due to its non-selective nature of EGFR inhibition, it accounts for a broader spectrum of anti-tumor activity with improved efficacy. This molecule binds to the ATP binding pocket of recombinant EGFR and HER2 protein kinase. It inhibits the recombinant EGFR and HER-2 tyrosine kinases by 50% (IC₅₀) at concentrations of 10.8 and 9.3 nmol/L, respectively, thus preventing auto phosphorylation and subsequent inhibition of downstream signalling.

Canertinib (CI-1033) is a 3-chloro 4-fluoro 4-anilinoquinazoline compound. It is an orally active low-molecular-weight irreversible pan-EGFR family tyrosine kinase inhibitor. It is a new generation tyrosine kinase inhibitor, designed to alkylate a cysteine residue specific to ErbB family receptors, resulting in irreversible inhibition of these receptors and their downstream mitogenic signalling pathways. Canertinib binds to the ATP binding pocket and the acrylamide side chain at carbon 6, and closely associates with cysteine residue 773 of EGFR and residues 784 and 778 of HER2 and HER4, respectively, resulting in their permanent inactivation. It also effectively inhibits HERS-dependent signalling due to the unavailability of partner receptors for heterodimerization of EGFR family members. Canertinib also induces ubiquitination and endocytosis of the receptors. Growth inhibition and apoptosis can be achieved in 1 micromolar or nano molar range and this selectivity explains the minimal toxicity observed in multi-dose animal studies.

It will be appreciated that other inhibitors of EGFR tyrosine kinase activity may be used in accordance with invention.

In other embodiments, EGFR signalling may be inhibited by inhibiting binding of ligand to an extracellular binding domain of EGFR. Monoclonal antibodies that block binding of ligand to the extracellular ligand-binding domain of EGFR prevent receptor dimerization, auto-phosphorylation and downstream signalling. Such antibodies also induce receptor internalization, ubiquitinization, degradation and prolonged downregulation. Examples of monoclonal antibodies that bind to the EGFR extracellular domain and block ligand binding include cetuximab, panitumumab, zalutumumab, nimotuzumab, and matuzumab.

Inhibition of EGFR signalling reduces astrocyte and microglial activation, and inhibits the TLR/IL-1R response to IL-1β and TLR ligands.

In one embodiment of the invention, EGFR signalling is inhibited so as to inhibit phosphorylation of HDAC6 by the EGFR. This may be achieved, for example, by inhibiting the intracellular tyrosine kinase of EGFR, or by inhibiting binding of ligand to the EGFR. HDAC6 has been shown to deacetylate TDP43 (Cohen et al., Nat Commun. 2015 Jan. 5; 6:5845). EGFR-mediated phosphorylation of HDAC6 (at Tyr 570) inhibits HDAC6 deacetylase activity (Deribe et al., Sci Signal. 2009 Dec. 22; 2(102):ra84), so blockade of the EGFR should increase HDAC6 activity and thus TDP43 deacetylation, thereby reducing TDP43 aggregation.

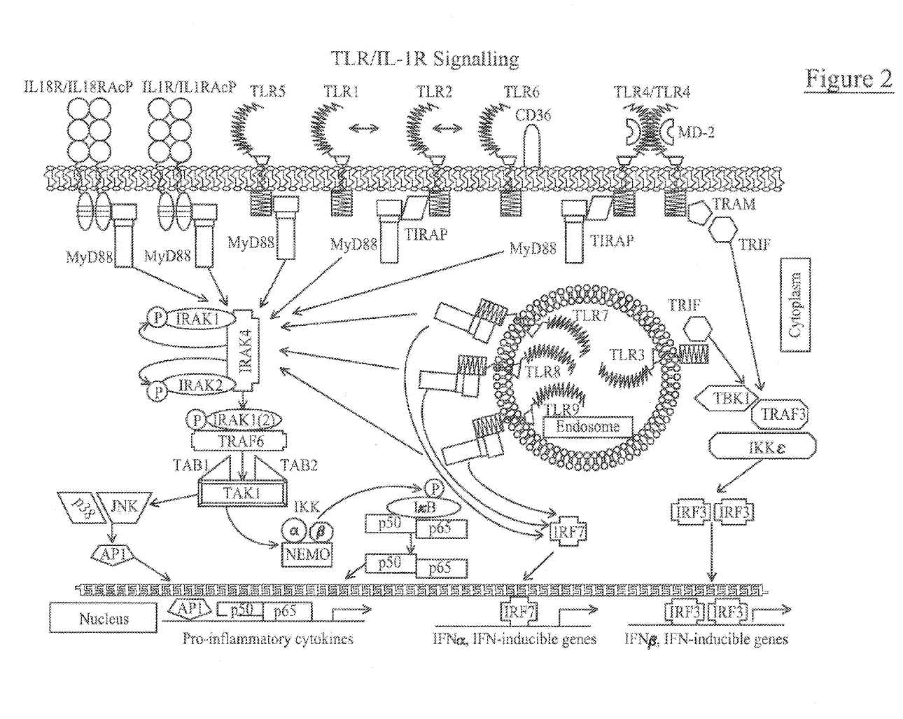

TLR/IL-1R signalling pathways are illustrated in FIG. 2 (taken from Loiarro et al., Mediators of Inflammation, 2010, Article ID 674363). Upon recognition of their cognate ligands, TLR/IL-1R proteins homo- or hetero dimerize (TLR1/2, TLR2/6, IL-1R/IL-1RacP) and initiate a signalling cascade through recruitment of different combinations of TIR-domain-containing adaptor protein (namely, MyD88, MAL/TIRAP, TRIF, and TRAM) to their TlR domain. All receptors of the superfamily, with the exception of TLR3, use MyD88 to initiate their signalling pathway. In some cases, MyD88 acts in concert with other adaptors, like MAL/TIRAP in the response triggered by stimulation of TLR4, TLR1/2, and TLR2/6. TLR3-mediated signalling requires only the adaptor molecule TRIF, which is also recruited by TLR4 in association with the other adaptor TRAM.

The TLR/IL-1R-induced pathways can be sub-grouped in two classes: MyD88-dependent and MyD88-independent responses. In the MyD88-dependent pathway, MyD88 associates with IRAK4, IRAK1 and/or IRAK2. IRAK4 in turn phosphorylates IRAK1 and IRAK2 and promotes their association with TRAF6, which serves as a platform to recruit the kinase TAK1. Once activated, TAK1 activates the IKK complex, composed of IKKα, IKKβ and NEMO (IKKγ), which catalyzes phosphorylation and subsequent degradation of IκB rendering NF-κB (i.e., p50/p65) free to translocate from the cytosol to the nucleus and activate NF-κB-dependent genes. TAK1 can also activate mitogen-activated protein kinases (MAPKs), such as p38 and JNK, leading to the activation of transcription factor AP-1. The concomitant activation of NF-κB and AP-1 induces a pleiotropic inflammatory response through the production of proinflammatory cytokines. The transcription factor IRF7 is also activated downstream of TLRs 7, 8, and 9, leading to its translocation into the nucleus and to activation of IFNα and IFN-inducible genes.

TLR3 and TLR4 both signal through the adaptor TRIF in the MyD88-independent pathway. TLR3 requires only TRIF as adaptor. Recruitment of TRAM is required to bridge TRIF to TLR4. Thus, TLR4 is capable of activating both MyD88-dependent and TRIF-dependent signalling pathways, in a sequential process that involves the endocytosis of the TLR4 complex. TLR4 first induces MAL/TIRAPMyD88 signalling at the plasma membrane. Then, following its endocytosis into early endosomes, TLR4 activates TRAM-TRIF signalling. TRIF interacts with TRAF3 to activate the noncanonical IKKs, TBK1, and IKKε resulting in the dimerization and activation of IRF3, which then translocates into the nucleus activating the transcription of IFNβ and IFN-inducible genes (Loiarro et al., Mediators of Inflammation, 2010, Article ID 674363).

The Applicant has recognised that rebalancing the IL-R/TLR signalling pathway towards IRF3 would be neuroprotective in neurodegenerative diseases, such as MNDs (and in particular, ALS). It is proposed that damage to motor neurons results in the release of DAMPs such as HMGB1, causing TLR4 receptor activation, enhancement of inflammosome activation, and thus increased IL-1β production by microglia. IL-1β-mediated activation of the TLR/IL-1 signalling pathway generates more NFkB and thus more IL-113, thus generating a self-maintained cycle of IL-1β generation which can induce motoneuron necrosis (Brites and Vaz, Front Cell Neurosci. 2014 May 22; 8:117). In microglia and astrocytes of the mSOD1 mouse, elevated NFkB activity results in associated motoneuron necrosis (Frakes et al., Neuron. 2014 Mar. 5; 81(5):1009-23). In the brain, reduced NFkB activity is associated with reduced post MCAO neuronal damage (Vartanian et al., J Neuroinflammation. 2011 Oct. 14; 8:140). In motoneurons necroptosis is mediated through Rip-1, which increases NFkB production, suggesting that targeting NFkB could be a therapeutic option. However the universal and multi-functional properties of this transcription factor make this difficult.

The Applicant has recognised that the increased levels of IL-1β in the spinal cord of SOD1 mice, the ability of mSOD1 to increase IL-1β production, and the fact that such IL-1β accelerates ALS progression (Meissner et al., Proc Natl Acad Sci USA. 2010 Jul. 20; 107(29):13046-50), suggests that modulation of IL-1β and the IL-1R signalling pathways would be beneficial for the prevention and treatment of neurodegenerative diseases, such as the MNDs, and ALS in particular. In addition, the apparent requirement for active EGFR to generate the LPS response in microglia (Qu et al., 2012) suggests that blockade of the EGFR could reduce TLR-stimulated inflammation.

Haplo-insufficiency in TBK1, a central component of the TRIF pathway, has been shown to be sufficient to cause fALS (Freischmidt et al., Nat Neurosci. 2015 May; 18(5):631-6). The TRIF pathway is part of the IL-1R/TLR route to IRF3 production, and IRF3 is generally neuroprotective. Indeed neuroprotection through preconditioning drives the TLR/IL-1 signalling pathway away from NFkB and towards IRF3 activation (Vartanian et al., 2011). We have recognised that rebalancing the IL-R/TLR signalling pathway towards IRF3 would be neuroprotective in neurodegenerative diseases, such as the MNDs (for example ALS). In particular, we have appreciated that inhibition of MyD88-dependent TLR/IL-R1 signalling will result in preferential IRF3 generation following IL-1/TLR stimulation. This will reduce the drive to NFkB, and thus reduce IL-1β production, glial activation and motoneuron death.

Thus, according to certain embodiments of the invention, MyD88-dependent TLR/IL-R1 signalling is inhibited so as to inhibit production of IL-1β and NFkB.

Inhibition of MyD88-dependent TLR/IL-R1 signalling may be achieved by inhibition of the Myddosome (the myddosome is an oligomeric signaling complex consisting of the adaptor protein MyD88 and IRAK4 kinase), for example by inhibiting the myddosome kinases IRAK1 and/or IRAK4.

The Applicant has recognised that the observed association of IRAK1 with TDP43 (Li et al., 2014, supra) may mediate the priming phosphorylation of TDP43, triggering the aggregation, and cytoplasmic accumulation of this protein, resulting in functional aberration. Thus, inhibition of MyD88-dependent TLR/IL-R1 signalling, in particular by inhibiting IRAK1 kinase activity, or by inhibiting MyD88-dependent TLR/IL-R1 signalling upstream of phosphorylation of TDP43 by IRAK1, is also expected to inhibit phosphorylation of TDP43, and thus inhibit the formation of inclusions of TDP43.

IRAK1 and/or IRAK4 may be inhibited by administering a small molecule inhibitor of IRAK1 and/or IRAK4 to the subject. Examples of suitable inhibitors of IRAK1 are listed in the table below, with their affinities. In particular embodiments of the invention, the inhibitor of IRAK1 and/or IRAK4 kinase activity is gefitinib.

TABLE 2
IRAK1 inhibitors

| | Affinity | | Affinity |
|---|---|---|---|
| Ligand | (−log₁₀) | Units | (nM) |
| lestaurtinib | 8.2 | pK_{d} | 6.1 |
| tamatinib | 8.0 | pK_{d} | 9.7 |
| sunitinib | 7.8 | pK_{d} | 14.0 |
| SU-14813 | 7.6 | pK_{d} | 26.0 |
| staurosporine | 7.4 | pK_{d} | 40.0 |
| NVP-TAE684 | 7.3 | pK_{d} | 45.0 |
| KW-2449 | 7.3 | pK_{d} | 48.0 |
| crizotinib | 7.3 | pK_{d} | 49.0 |
| gefitinib | 7.2 | pK_{d} | 69.0 |
| AST-487 | 6.9 | pK_{d} | 130.0 |
| dovitinib | 6.8 | pK_{d} | 170.0 |
| JNJ-28312141 | 6.7 | pK_{d} | 220.0 |
| fedratinib | 6.7 | pK_{d} | 220.0 |
| afatinib | 6.6 | pK_{d} | 240.0 |
| ruxolitinib | 6.5 | pK_{d} | 290.0 |
| canertinib | 6.3 | pK_{d} | 450.0 |
| alvocidib | 6.3 | pK_{d} | 540.0 |
| bosutinib | 6.2 | pK_{d} | 600.0 |
| imatinib | 5.9 | pK_{d} | 1200.0 |
| vandetanib | 5.9 | pK_{d} | 1200.0 |
| PHA-665752 | 5.8 | pK_{d} | 1600.0 |
| BI-2536 | 5.7 | pK_{d} | 1900.0 |
| neratinib | 5.6 | pK_{d} | 2300.0 |
| tandutinib | 5.6 | pK_{d} | 2700.0 |

P-glycoprotein (P-gp) is a transmembrane efflux pump, and is the most important drug transporter for reducing the entry of drugs into the CNS. In some embodiments, an inhibitor of EGFR signalling (in particular, a small molecule inhibitor of EGFR tyrosine kinase) and/or an inhibitor of MyD88-dependent TLR/IL-1R signalling (in particular, a small molecule inhibitor of IRAK1 and/or IRAK4) may be co-administered, or administered sequentially with a P-gp inhibitor (such as cyclosporine A, ketoconazole, quinidine, ritonavir, verapamil, everolimus, or elacridar (GF120918)) to increase CNS exposure of the signalling inhibitor(s).

According to one embodiment, inhibition of EGFR signalling and inhibition of MyD88-dependent TLR/IL-R1 signalling is achieved by administration of gefitinib (Iressa) to the subject. Gefitinib is an EGFR tyrosine kinase inhibitor, and also inhibits kinase activity of IRAK1 and IRAK4 in the MyD88-dependent TLR/IL-R1 signalling pathway. Thus, administration of gefitinib would:
Block EGFR signalling thus:
   Reducing astrocyte and microglial activation
   Inhibiting the TLR/IL-R1 response to IL-1β and TLR ligands
   Increasing HDAC6 activity thus reducing the acetylation status (and thus inhibiting aggregation) of TDP43
Inhibit MyD88-dependent TLR/IL-R1 signalling thus:
   Rebalancing the TLR/IL-R1 signalling pathway towards IRF3 production (and away from NFkB production), consistent with neuroprotection
   Reducing NFkB production, inflammation and necroptosis
   Inhibiting the phosphorylation (and thus aggregation) of TDP43 via inhibition of IRAK1 kinase activity

Gefitinib is a P-glycoprotein (P-gp) substrate (see Togashi et aL, Cancer Chemother Pharmacol. 2012 September; 70(3):399-405). In some embodiments, gefitinib is co-administered, or administered sequentially with a P-gp inhibitor (such as cyclosporine A, ketoconazole, quinidine, ritonavir, verapamil, everolimus, or elacridar (GF120918)) to increase CNS exposure (as it does in mice: Chen et al., Lung Cancer. 2013 November; 82(2):313-8).

According to the invention there is provided a pharmaceutical composition, which comprises an inhibitor of EGFR signalling, and an inhibitor of MyD88-dependent TLR/IL-R1 signalling, and a pharmaceutically acceptable carrier, excipient, or diluent, wherein the inhibitor of EGFR signalling, and an inhibitor of MyD88-dependent TLR/IL-R1 signalling are different compounds.

According to the invention there is also provided a combined preparation, which comprises: (a) an inhibitor of EGFR signalling; and (b) an inhibitor of MyD88-dependent TLR/IL-R1 signalling, wherein the inhibitor of EGFR signalling, and the inhibitor of MyD88-dependent TLR/IL-R1 signalling are different compounds.

There is further provided according to the invention a pharmaceutical composition, or a combined preparation, of the invention, which further comprises a P-glycoprotein inhibitor.

There is also provided according to the invention a composition, which comprises an inhibitor of EGFR signalling, an inhibitor of MyD88-dependent TLR/IL-R1 signalling, and a P-glycoprotein inhibitor.

There is also provided according to the invention a pharmaceutical composition, which comprises an inhibitor of EGFR signalling, an inhibitor of MyD88-dependent TLR/IL-R1 signalling, a P-glycoprotein inhibitor, and a pharmaceutically acceptable carrier, excipient, or diluent.

According to the invention there is also provided a combined preparation, which comprises: (a) an inhibitor of EGFR signalling; (b) an inhibitor of MyD88-dependent TLR/IL-R1 signalling; and (c) a P-glycoprotein inhibitor.

The inhibitor of EGFR signalling, and the inhibitor of MyD88-dependent TLR/IL-R1 signalling may be the same compound, or different compounds. In some embodiments, the inhibitor of EGFR signalling, and the inhibitor of MyD88-dependent TLR/IL-R1 signalling may be gefitinib. In other embodiments (in particular, where the inhibitor of EGFR signalling, and the inhibitor of MyD88-dependent TLR/IL-R1 signalling are the same compound) the inhibitor of EGFR signalling, and the inhibitor of MyD88-dependent TLR/IL-R1 signalling may exclude gefitinib.

The inhibitor of EGFR signalling, the inhibitor of MyD88-dependent TLR/IL-R1 signalling, and the P-glycoprotein inhibitor may be administered together (i.e. co-administered) or sequentially, in any order. In particular embodiments, the P-glycoprotein inhibitor is administered before the inhibitor of EGFR signalling and the inhibitor of MyD88-dependent TLR/IL-R1 signalling.

There is also provided according to the invention a composition, which comprises gefitinib, and a P-glycoprotein inhibitor.

There is also provided according to the invention a pharmaceutical composition, which comprises gefitinib, and a P-glycoprotein inhibitor, and a pharmaceutically acceptable carrier, excipient, or diluent.

There is further provided a combined preparation, which comprises: (a) gefitinib; and (b) a P-glycoprotein inhibitor.

The P-glycoprotein inhibitor may be selected from the group consisting of cyclosporine A, ketoconazole, quinidine, ritonavir, verapamil, everolimus, or elacridar (GF120918), for example quinidine. In particular embodiments, the P-glycoprotein inhibitor is elacridar.

There is further provided according to the invention a composition, a pharmaceutical composition, or a combined preparation of the invention for use in the prevention or treatment of a neurodegenerative disease.

There is also provided according to the invention use of a composition, a pharmaceutical composition, or a combined preparation of the invention in the manufacture of a medicament for the prevention or treatment of a neurodegenerative disease.

There is also provided according to the invention a method of preventing or treating a neurodegenerative disease, which comprises administering effective amounts of gefitinib and a P-glycoprotein inhibitor to a subject in need of such prevention or treatment.

The gefitinib and the P-glycoprotein inhibitor may be co-administered, or administered sequentially.

The neurodegenerative disease may be a motor neuron disease, such as amyotrophic lateral sclerosis (ALS).

The neurodegenerative disease may be a familial or sporadic neurodegenerative disease. In particular embodiments, the neurodegenerative disease (in particular motor neurone disease, such as ALS) is a familial neurodegenerative disease. In other particular embodiments, the neurodegenerative disease (in particular motor neurone disease, such as ALS) is a sporadic neurodegenerative disease.

The components of a combined preparation of the invention may be for simultaneous, separate, or sequential use.

The term “combined preparation” as used herein refers to a “kit of parts” in the sense that the combination components (a) and (b) can be dosed independently or by use of different fixed combinations with distinguished amounts of the combination components (a) and (b). The components can be administered simultaneously or one after the other. If the components are administered one after the other, preferably the time interval between administration is chosen such that the effect on the treated disorder or disease in the combined use of the components is greater than the effect which would be obtained by use of only any one of the combination components (a) and (b).

The components of the combined preparation may be present in one combined unit dosage form, or as a first unit dosage form of component (a) and a separate, second unit dosage form of component (b). The ratio of the total amounts of the combination component (a) to the combination component (b) to be administered in the combined preparation can be varied, for example in order to cope with the needs of a patient sub-population to be treated, or the needs of the single patient, which can be due, for example, to the particular disease, age, sex, or body weight of the patients.

Preferably, there is at least one beneficial effect, for example an enhancing of the effect of one of the components, or a mutual enhancing of the effect of the combination components (a) and (b), for example a more than additive effect, additional advantageous effects, fewer side effects, less toxicity, or a combined therapeutic effect compared with a non-effective dosage of one or both of the combination components (a) and (b), and very preferably a synergism of the combination components (a) and (b).

In some embodiments of the invention, the neurodegenerative disease is a motor neuron disease, such as ALS, PLS, PMA, PBP, or Pseudobulbar palsy, or Alzheimer's disease, or Parkinson's disease, or fronto temporal dementia (FTI).

As used herein, the terms “treatment”, “treating”, “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting or slowing its development; and (c) relieving the disease, i.e., causing regression of the disease.

The term “subject” used herein includes any human or nonhuman animal. The term “nonhuman animal” includes all mammals, such as nonhuman primates, sheep, dogs, cats, cows, horses.

It will be appreciated that, in methods of the invention, the subject should be administered with a therapeutically effective amount of an inhibitor of EGFR signalling and an inhibitor of MyD88-dependent TLR/IL-R1 signalling (and a P-glycoprotein inhibitor, where appropriate).

A “therapeutically effective amount” refers to the amount of an inhibitor of EGFR signalling and an inhibitor of MyD88-dependent TLR/IL-R1 signalling that, when administered to a subject for treating a disease, is sufficient to effect such treatment for the disease. The “therapeutically effective amount” will vary depending on the inhibitor(s) used, the disease and its severity and the age, weight, etc., of the subject to be treated.

For example, a therapeutically effective amount of gefitinib is 100-750 mg per day when co-administered, or administered sequentially with a P-gp inhibitor. In some embodiments, a therapeutically effective amount of gefitinib may also be 100-750 mg per day when administered without a P-gp inhibitor, for example when administered directly to the CNS (such as directly to the brain or spinal cord).

An inhibitor of EGFR signaling, and an inhibitor of MyD88-dependent TLR/IL-R1 signalling, may be administered to a subject using any available method and route suitable for drug delivery to the CNS, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intrathecal, and intravenous routes, i.e., any route of administration other than through the alimentary canal. Parenteral administration can be carried to effect systemic or local delivery. Where systemic delivery is desired, administration typically involves invasive or systemically absorbed topical or mucosal administration of pharmaceutical preparations. Enteral routes of administration include, but are not necessarily limited to, oral and rectal (e.g., using a suppository) delivery.

Conventional and pharmaceutically acceptable routes of administration include intranasal, intramuscular, intra-tracheal, intrathecal, intracranial, subcutaneous, intradermal, topical, intravenous, intraperitoneal, intra-arterial (for example, via the carotid artery), spinal or brain delivery, rectal, nasal, oral, and other enteral and parenteral routes of administration.

In some embodiments, an inhibitor of EGFR signalling and/or an inhibitor of MyD88-dependent TLR/IL-1R signalling is administered by injection and/or delivery, for example, to a site in a brain artery or directly into brain tissue.

In particular embodiments, an inhibitor of EGFR signalling and/or an inhibitor of MyD88-dependent TLR/IL-1R signalling is administered by direct delivery to the CNS, in particular in to the spinal cord or brain, such as by intracerebroventricular (ICV) administration. Direct administration in to the brain can be undertaken in combination with a controlled delivery device, such as an in-dwelling cannula or pump (for example, implanted subcutaneously at a suitable location). Suitable methods of ICV administration to human subjects are described, for example, in Paul et al., J an Invest. 2015; 125(3):1339-1346).

A composition of the invention may be provided in a formulation suitable for, or adapted for, administration directly to the CNS, in particular in to the spinal cord or brain, for example of a human subject. In some embodiments, the formulation comprises one or more electrolytes present in endogenous CSF. In particular embodiments, the one or more electrolytes are selected from sodium, potassium, calcium, magnesium, phosphorous, and chloride ions. In a particular embodiment, the formulation comprises a solution that closely matches the electrolyte concentrations of endogenous CSF of the subject to be treated, for example a human subject. For example, in particular embodiments the formulation comprises a solution comprising any (or each) of: 100-200 mM sodium ion; 1-5 mM potassium ion; 1-2 mM calcium ion; 0.5-1.5 mM magnesium ion; 0.5-1.5 mM phosphorous ion; and 100-200 mM chloride ion. For example, in a particular embodiment the formulation comprises a solution comprising 150 mM sodium ion, 3 mM potassium ion, 1.4 mM calcium ion, 0.8 mM magnesium ion, 1.0 mM phosphorous ion, and 155 mM chloride ion.

In particular embodiments, a composition of the invention suitable for, or adapted for, administration directly to the CNS, in particular in to the spinal cord or brain, for example of a human subject, does not include a P-glycoprotein inhibitor.

The inhibitor(s) may be administered in a single dose or in multiple doses. A suitable frequency of administration may be at least once per day, every other day, once per week, once every two, three, or four weeks, once every month, two months, or once every three to six months. For example, given its half-life, a suitable frequency of administration of gefitinib is at least once per day. The inhibitor(s) may be administered over a period of at least a week, at least a month, at least three to six months, at least one, two, three, four, or five years, or over the course of the disease, or the lifetime of the subject.

Where the inhibitor of EGFR signalling, and the inhibitor of MyD88-dependent TLR/IL-R1 signalling are different compounds, they may be co-administered, or administered sequentially. If the inhibitors are administered sequentially, they may be administered in any order. It will be appreciated that the second inhibitor to be administered should be administered whilst the first inhibitor remains effective. The timing of sequential administration will depend on various factors, such as the respective half-lives of the inhibitors, and their bioavailability. Typically, however, it is expected that the inhibitors should be administered within 96, 72, 48, 36, 24, 12, 6, 5, 4, 3, 2, or 1 hours of each other.

Where a P-gp inhibitor is used, this may be co-administered with the inhibitor(s) of EGFR and MyD88-dependent TLR/IL-R1 signalling, or the P-gp inhibitor and the inhibitor(s) of EGFR and MyD88-dependent TLR/IL-R1 signalling may be administered sequentially, for example within 96, 72, 48, 36, 24, 12, 6, 5, 4, 3, 2, or 1 hours of each other (i.e. the P-gp inhibitor may be administered before, or after the inhibitor(s) of EGFR and MyD88-dependent TLR/IL-R1 signalling). For example, if the inhibitor of EGFR and MyD88-dependent TLR/IL-R1 signalling is gefitinib, the P-gp inhibitor and the gefitinib may be co-administered, or administered sequentially, for example within 96, 72, 48, 36, 24, 12, 6, 5, 4, 3, 2, or 1 hours of each other. The P-gp inhibitor may be administered before the gefitinib, or the gefitinib may be administered before the P-gp inhibitor. In particular embodiments, the P-gp inhibitor is elacridar.

The amount of P-gp inhibitor that is co-administered, or administered sequentially with the inhibitor of EGFR signalling and the inhibitor of MyD88-dependent TLR/IL-R1 signalling is likely to depend on the particular inhibitors used. However, a person of ordinary skill in the art can readily determine the appropriate amount of each inhibitor to administer to ensure that a therapeutically effective amount of the inhibitor of EGFR signalling and the inhibitor of MyD88-dependent TLR/IL-R1 signalling penetrates the CNS of the subject to be treated, For example, based on the results obtained in Example 3 below, it is expected that a therapeutically effective amount of gefitinib for a human subject is 100-750 mg per day when co-administered, or administered sequentially with 100 mg per day of the P-gp inhibitor Elacridar.

Methods of preparing pharmaceutical compositions are known, or will be apparent, to those skilled in the art. See, e.g., Remington's Pharmaceutical Sciences, Mack Publishing Company, Easton, Pa., 17th edition, 1985.

Compositions of the invention can be formulated into pharmaceutical compositions by combination with appropriate pharmaceutically acceptable carriers, pharmaceutically acceptable diluents, or pharmaceutically acceptable excipients, and can be formulated into preparations in solid, semi-solid, liquid or gaseous forms, such as tablets, capsules, powders, granules, solutions, injections, inhalants and aerosols.

Pharmaceutically acceptable carriers, excipients, or diluents may include, for example: water, saline, dextrose, glycerol, ethanol, a salt, e.g., NaCl, MgCl₂, KCl, MgSO₄, etc.; a buffering agent, e.g., a phosphate buffer, a citrate buffer, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; glycerol; and the like.

Pharmaceutically acceptable carriers, excipients and diluents are nontoxic to recipients at the dosages and concentrations employed, and can for example include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

For oral preparations, a pharmaceutical composition of the invention may include appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Pharmaceutical compositions for injection can be formulated by dissolving, suspending or emulsifying the active ingredients in an aqueous or nonaqueous solvent, such as vegetable or other similar oils, propylene glycol, synthetic aliphatic acid glycerides, injectable organic esters (e.g., ethyl oleate), esters of higher aliphatic acids or propylene glycol; and if desired, with conventional additives such as solubilizers, isotonic agents, suspending agents, emulsifying agents, stabilizers and preservatives. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Typically, injectable compositions are prepared as liquid solutions or suspensions; solid forms suitable for solution in, or suspension in, liquid vehicles prior to injection can also be prepared.

The pharmaceutical composition can be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents can be used for the production of pharmaceutical compositions for parenteral administration; see also Chen (1992) Drug Dev Ind Pharm 18, 1311-54.

A tonicity agent can be included in the formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions can be suitable. The term “isotonic” denotes a solution having the same tonicity as some other solution with which it is compared, such as a physiological salt solution or serum.

Embodiments of the invention are described below, by way of example only, with reference to the accompanying drawings in which:

FIG. 1 shows a schematic illustration of EGFR signalling;

FIG. 2 shows a schematic illustration of TLR/IL-1R signalling;

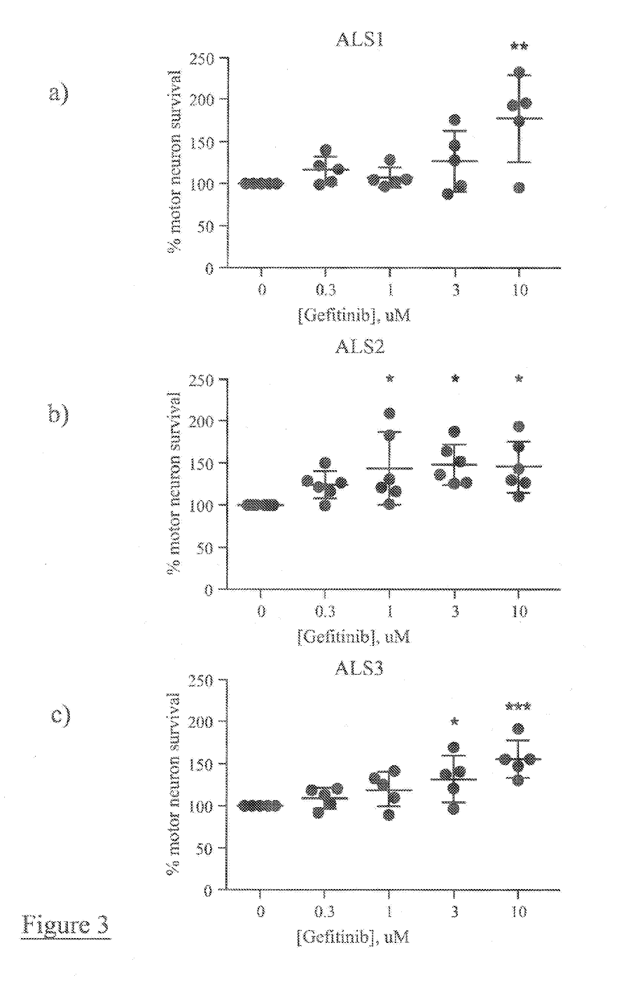

FIG. 3 shows the effect of gefitinib in an in vitro model in which iAstrocytes derived from fibroblasts of three different ALS patients (ALS1, ALS2, and ALS3) were co-cultured with mouse motor neurons. ALS iAstrocytes were pre-treated with various concentrations of Gefitinib for 24 hours, prior to murine Hb9-GFP motor neuron seeding in co-culture. The number of viable motor neurons was then measured 24 and 72 hours after motor neuron seeding, and the percentage motor neuron survival was calculated, and then normalised to the untreated control of the respective line. *P<0.05, “P<0.01, ***P<0.001. One-way ANOVA with Dunnett's post-hoc test. Data are mean±SD. n=5-6; and

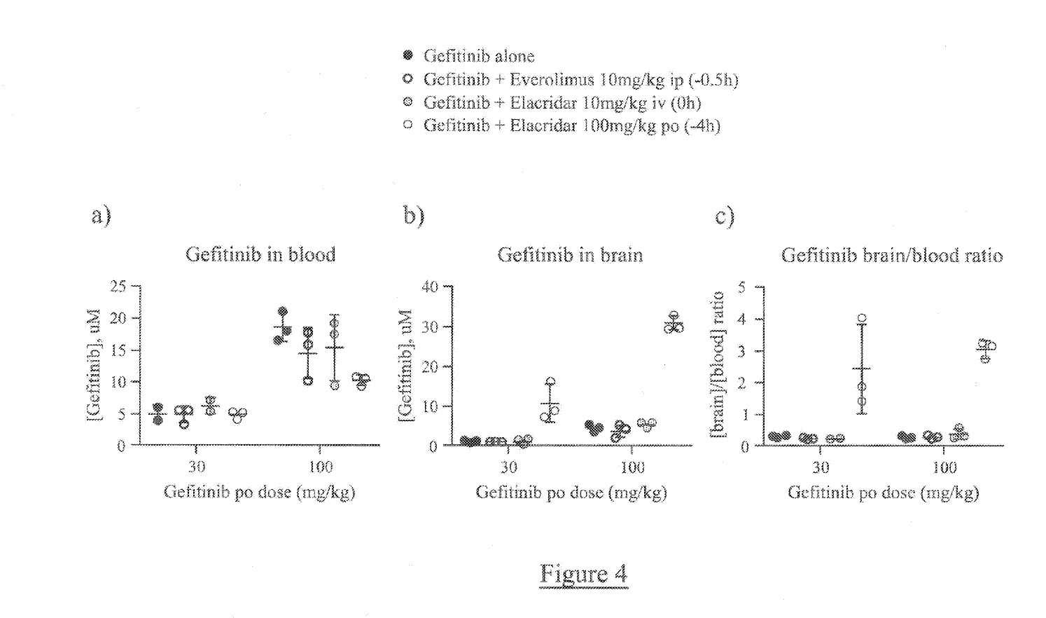

FIG. 4 shows the results of analysis of entry of Gefitinib into the brain in C57BL/6 mice following oral administration of Gefitinib with prior administration of different P-gp inhibitors. The figures show Gefitinib levels in blood (μM) (a) and brain (μM) (b), and the brain to blood ratio of Gefitinib concentration (c), two hours post oral dose. For each dose of Gefitinib (30 or 100 mg/kg po), the figures show (in order from left to right): Gefitinib alone; Gefitinib+Everolimus 10 mg/kg ip (−0.5 h); Gefitinib+Elacridar 10 mg/kg iv (Oh); and Gefitinib+Elacridar 100 mg/kg po (−4 h).

### EXAMPLE 1

### Treatment of ALS

250 mg of gefitinib is co-administered once per day with 600 mg quinidine (a P-gp inhibitor) to a human subject suffering from ALS. Administration of 250 mg gefitinib results in a plasma concentration of approximately 250 nM. In mice such a plasma concentration is associated with a total brain concentration of approximately 100 nM. Co-administration of gefitinib with quinidine increases the brain exposure to gefitinib. Administration of such amount of gefitinib is expected to be sufficient to inhibit EGFR and IRAK1 based on its pharmacological properties:
Ki at EGFR: 1 nM
Ki at IRAK1: 70 nM
Ki at IRAK4: 500 nM

### EXAMPLE 2

### Effect of Gefitinib in an In Vitro Model of ALS

This example describes the effect of Gefitinib on motor neuron survival in an in vitro model of ALS. This model uses human fibroblast-derived astrocytes and mouse Hb9-GFP+ motor neurons in co-culture (Meyer et al., 2014, PNAS 111, 829-832). The fibroblasts were reprogrammed to induced neural progenitor cells (iNPCs), which were differentiated into Astrocytes. Astrocytes derived from ALS patients cause death of the wild-type Hb9-GFP+ mouse motor neurons in co-culture, a property not seen in astrocytes derived from normal (non-ALS) patients. Interestingly the ALS astrocytes display some abnormalities in metabolism and oxidative stress, which are increased by 10-15 fold in the presence of motor neurons.

Materials and Methods

iNPCs were derived from ALS patient fibroblasts as previously described (Meyer et a/. 2014, PNAS 111, 829-832), and were differentiated into iAstrocytes by culturing in supplemented DMEM (Sigma) (10% (vlv) FBS (Sigma), 50units/ml penicillin/streptomycin (Lonza), 1X N-2 supplement (Thermo-Fisher Scientific) for at least 5 days. Murine Hb9-GFP+ motor neurons were differentiated from murine Hb9-GFP+ embryonic stem cells via embryoid bodies, as previously described (Haidet-Phillips et al. 2011, Nature Biotechnology 29, 824-828; Wichterle et al. 2002, Cell 110, 385-397).

3,000 human iAstrocytes were seeded per well on fibronectin-coated 384-well plates. 24 hours later, gefitinib (Cayman Chemical Company, cat. #13166) was delivered in 100% drug-grade DMSO to iAstrocyte media using an Echo550 liquid handler (Labcyte). The final concentration of DMSO was 0.24% (v/v) in the media in all wells. Plates were centrifuged at 1,760×g for 60 s. 24 hours later, 2,000 murine Hb9-GFP+ motor neurons were seeded per well in motor neuron media (KnockOut DMEM (45% vlv), F12 medium (45% v/v), KO Serum Replacement (10% v/v), 50units/ml penicillin/streptomycin (Lonza), 1mM L-glutamine, 1× N-2 supplement (Thermo-Fisher Scientific), 0.15% filtered glucose, 0.0008% (v/v) 2-mercaptoethanol, 20 ng/ml GDNF, 20 ng/ml BDNF, 20 ng/ml CNTF) and co-cultured on top of the pre-treated iAstrocytes. Plates were centrifuged at 1,760 x g for 60 s. Hb9-GFP+ motor neurons were imaged after 24 and 72 hours using an INCELL analyser 2000 (GE Healthcare), and the number of viable motor neurons was counted using the INCELL analyser software (GE Healthcare).

The number of viable motor neurons (defined as GFP+ motor neurons with at least 1 axon) that survived after 72 hours in co-culture was calculated as a percentage of the number of viable motor neurons after 24 hours in co-culture. Percentage survival of motor neurons was then normalised to the DMSO control for each individual iAstrocyte line. One-way ANOVA with Dunnett's post hoc test was performed.

Results

The results, plotted in FIG. 3, show that gefitinib promotes a dose-dependent increase in motor neuron survival in co-cultures from three different patients, suggesting that gefitinib will have beneficial effects in patients with ALS.

Conclusions

It was concluded from these results that the toxic nature of ALS patient-derived astrocytes, as revealed by decreased motor neuron survival in ALS astrocyte/motor neuron co-cultures, is reduced by gefitinib. Although not wishing to be bound by theory, these results are consistent with inhibition of the EGFR and IRAK 1 by gefitinib. Such inhibition is expected to inhibit the generation of Myddosome-driven NFkB, thereby protecting motor neurons.

### EXAMPLE 3

### Penetration of Gefitinib into the CNS in the Presence of a P-gp Inhibitor

For Gefitinib to be effective in the treatment of ALS, it is important that it enters the CNS. This example describes the results of analysis of entry of Gefitinib into the brain in C57BL/6 mice following oral administration of Gefitinib with prior administration of different P-gp inhibitors.

Mice were given a single dose of either Everolimus (10 mg/kg ip, −0.5 h), Elacridar (10 mg/kg iv, Oh), or Elacridar (100 mg/kg po), or no dose. Mice were then given a single oral dose of Gefitinib at either 30 mg/kg po or 100 mg/kg po. Blood and brain were collected 2 h after Gefitinib administration, and Gefitinib levels were measured in blood and brain using ultra-high performance liquid chromatography coupled with time-of-flight mass spectrometry UHPLC-TOF-MS. The results are plotted in FIG. 4, which shows Gefitinib levels in blood (a), brain (b), and the ratio of Gefitinib brain concentration to Gefitinib blood concentration (c). n=3. Individual points, as well as mean and SD are shown.

The results show that the P-gp inhibitor Elacridar, administered at 100 mg/kg po, significantly increased the CNS penetration of Gefitinib at both the lower dose of Gefitinib (30mg/kg po) and the higher dose of Gefitinib (100mg/kg po). Gefitinib has an IC₉₀ for IRAK1 of approximately 0.7 WI, and an IC₉₀ for the EGFR of approximately 10 nM (IC₉₀: the concentration at which the enzyme activity is inhibited by 90%). It was concluded from the results that administration of Elacridar prior to Gefitinib, in mice, resulted in a concentration of Gefitinib in the CNS that is sufficient to inhibit both the EGFR and IRAK1.

Surprisingly, the P-gp inhibitor Everolimus increased the plasma exposure of Gefitinib, but did not appear to have any effect on CNS penetration of Gefitinib in C57BL/6 mice at the dose of 10 mg/kg ip. The reasons for this are not fully understood, although it is possible that Everolimus did not inhibit the blood brain barrier transporter responsible for excluding gefitinib from the brain.

1. A method of preventing or treating a neurodegenerative disease, which comprises inhibiting EGFR signalling, and inhibiting MyD88-dependent TLR/IL-R1 signalling, in the central nervous system of a subject in need of such prevention or treatment.

2. A method according to claim 1, wherein EGFR signalling and MyD88-dependent TLR/IL-R1 signalling are inhibited in microglial cells, astrocytes, or neurons of the subject.

3. A method according to claim 1 or 2, wherein EGFR signalling is inhibited so as to inhibit microglial cell activation and/or formation of inclusions of TDP43.

4. A method according to claim 3, wherein formation of inclusions of TDP43 is inhibited by inducing deacetylation of TDP43.

5. A method according to any preceding claim, wherein EGFR signalling is inhibited by inhibiting EGFR tyrosine kinase activity in the subject.

6. A method according to any preceding claim, wherein EGFR signalling is inhibited by administering an inhibitor of EGFR tyrosine kinase to the subject.

7. A method according to claim 6, wherein the tyrosine kinase inhibitor is a small molecule tyrosine kinase inhibitor selected from the group consisting of gefitinib, erlotinib, brigatinib, lapatinib, afatinib, and icotinib.

8. A method according to claim 6 or 7, wherein the EGFR tyrosine kinase inhibitor is co-administered, or administered sequentially to the subject with a P-glycoprotein inhibitor.

9. A method according to any of claims 1 to 4, wherein EGFR signalling is inhibited by inhibiting binding of ligand to an extracellular binding domain of EGFR.

10. A method according to any of claim 1 to 4, or 9, wherein EGFR signalling is inhibited by administering a monoclonal antibody that binds specifically to the extracellular binding domain of EGFR to the subject.

11. A method according to claim 10, wherein the monoclonal antibody is selected from the group consisting of cetuximab, panitumumab, zalutumumab, nimotuzumab, and matuzumab.

12. A method according to any preceding claim, wherein MyD88-dependent TLR/IL-R1 signalling is inhibited so as to inhibit production of IL-1β and NFkB and/or to inhibit formation of inclusions of TDP43.

13. A method according to claim 12, wherein formation of inclusions of TDP43 is inhibited by inhibiting phosphorylation of TDP43.

14. A method according to any preceding claim, wherein MyD88-dependent TLR/IL-R1 signalling is inhibited by inhibiting IRAK1 and/or IRAK4.

15. A method according to claim 14, wherein IRAK1 and/or IRAK4 is inhibited by administering a small molecule inhibitor of IRAK1 and/or IRAK4 to the subject.

16. A method according to claim 14 or 15, wherein the inhibitor is selected from the group consisting of lestaurtinib, tamatinib, sunitinib, SU-14813, staurosporine, NVP-TAE684, KW-2449, crizotinib, gefitinib, AST-487, dovitinib, JNJ-28312141, fedratinib, afatinib, ruxolitinib, canertinib, alvocidib, bosutinib, imatinib, vandetanib, PHA-665752, BI-2536, neratinib, and tandutinib.

17. A method according to claim 14 or 15, wherein the inhibitor is gefitinib.

18. A method according to any preceding claim, wherein EGFR signalling, and MyD88-dependent TLR/IL-R1 signalling are inhibited by administering gefitinib to the subject.

19. A method according to any preceding claim, wherein an inhibitor of EGFR signalling, and an inhibitor of MyD88-dependent TLR/IL-R1 signalling, is administered directly to the brain or spinal cord of the subject.

20. A method according to any of claims 15 to 18, wherein the inhibitor of IRAK1 and/or IRAK4 is co-administered, or administered sequentially to the subject with a P-glycoprotein inhibitor.

21. A method according to any preceding claim, wherein the neurodegenerative disease is a motor neuron disease.

22. A method according to claim 21, wherein the motor neuron disease is amyotrophic lateral sclerosis (ALS).

23. A method according to any preceding claim, wherein the neurodegenerative disease is a familial neurodegenerative disease.

24. An inhibitor of EGFR signalling, and an inhibitor of MyD88-dependent TLR/IL-R1 signalling, for use in the prevention or treatment of a neurodegenerative disease.

25. Use of an inhibitor of EGFR signalling, and an inhibitor of MyD88-dependent TLR/IL-R1 signalling, in the manufacture of a medicament for the prevention or treatment of a neurodegenerative disease.

26. Use according to claim 24 or 25, wherein the EGFR inhibitor inhibits EGFR signalling so as to inhibit microglial cell activation and/or formation of inclusions of TDP43.

27. Use according to any of claims 24 to 26, wherein the EGFR inhibitor inhibits formation of inclusions of TDP43 by inducing deacetylation of TDP43.

28. Use according to any of claims 24 to 27, wherein the EGFR inhibitor inhibits EGFR signalling by inhibiting EGFR tyrosine kinase activity.

29. Use according to any of claims 24 to 28, wherein the inhibitor of EGFR signalling is an inhibitor of EGFR tyrosine kinase.

30. Use according to claim 29, wherein the tyrosine kinase inhibitor is a small molecule tyrosine kinase inhibitor selected from the group consisting of gefitinib, erlotinib, brigatinib, lapatinib, afatinib, and icotinib.

31. Use according to any of claims 24 to 28, wherein the inhibitor of EGFR signalling inhibits binding of ligand to an extracellular binding domain of EGFR.

32. Use according to any of claim 24 to 28, or 31, wherein the inhibitor of EGFR signalling is a monoclonal antibody, or antigen-binding fragment thereof, that binds specifically to the extracellular binding domain of EGFR.

33. Use according to claim 32, wherein the monoclonal antibody is selected from the group consisting of cetuximab, panitumumab, zalutumumab, nimotuzumab, and matuzumab.

34. Use according to any of claims 24 to 33, wherein the inhibitor of MyD88-dependent TLR/IL-R1 signalling inhibits MyD88-dependent TLR/IL-R1 signalling so as to inhibit production of IL-1β and NFkB and/or to inhibit formation of inclusions of TDP43.

35. Use according to claim 34, wherein the inhibitor of MyD88-dependent TLR/IL-R1 signalling inhibits formation of inclusions of TDP43 by inhibiting phosphorylation of TDP43.

36. Use according to any of claims 24 to 35, wherein the inhibitor of MyD88-dependent TLR/IL-R1 signalling inhibits MyD88-dependent TLR/IL-R1 signalling by inhibiting IRAK1 and/or IRAK4.

37. Use according to claim 36, wherein the inhibitor of MyD88-dependent TLR/IL-R1 signalling is a small molecule inhibitor of IRAK1 and/or IRAK4.

38. Use according to claim 36 or 37, wherein the inhibitor is selected from the group consisting of lestaurtinib, tamatinib, sunitinib, SU-14813, staurosporine, NVP-TAE684, KW-2449, crizotinib, gefitinib, AST-487, dovitinib, JNJ-28312141, fedratinib, afatinib, ruxolitinib, canertinib, alvocidib, bosutinib, imatinib, vandetanib, PHA-665752, BI-2536, neratinib, and tandutinib.

39. Use according to claim 36 or 37, wherein the inhibitor is gefitinib.

40. Use according to any of claims 24 to 39, wherein the inhibitor of EGFR signalling, and of MyD88-dependent TLR/IL-R1 signalling is gefitinib.

41. Use according to any of claims 24 to 40, wherein the inhibitor of EGFR signalling, and the inhibitor of MyD88-dependent TLR/IL-R1 signalling, are administered directly to the brain or spinal cord.

42. Use according to any of claims 24 to 40, which further includes a P-glycoprotein inhibitor.

43. Use according to any of claims 24 to 42, wherein the neurodegenerative disease is a motor neuron disease.

44. Use according to claim 43, wherein the motor neuron disease is ALS.

45. Use according to any of claims 24 to 44, wherein the neurodegenerative disease is a familial neurodegenerative disease.

46. A pharmaceutical composition, which comprises an inhibitor of EGFR signalling, and an inhibitor of MyD88-dependent TLR/IL-R1 signalling, and a pharmaceutically acceptable carrier, excipient, or diluent, wherein the inhibitor of EGFR signalling, and the inhibitor of MyD88-dependent TLR/IL-R1 signalling are different compounds.

47. A combined preparation, which comprises: (a) an inhibitor of EGFR signalling; and (b) an inhibitor of MyD88-dependent TLR/IL-R1 signalling, wherein the inhibitor of EGFR signalling, and the inhibitor of MyD88-dependent TLR/IL-R1 signalling are different compounds.

48. A pharmaceutical composition according to claim 46, or a combined preparation according to claim 47, which further comprises a P-glycoprotein inhibitor.

49. A method according to claim 8, or 20, or a pharmaceutical composition, or a combined preparation according to claim 48, wherein the P-glycoprotein inhibitor is selected from the group consisting of cyclosporine A, ketoconazole, quinidine, ritonavir, verapamil, everolimus, and elacridar.

50. A composition, which comprises gefitinib, and a P-glycoprotein inhibitor.

51. A pharmaceutical composition, which comprises gefitinib, and a P-glycoprotein inhibitor, and a pharmaceutically acceptable carrier, excipient, or diluent.

52. A combined preparation, which comprises: (a) gefitinib; and (b) a P-glycoprotein inhibitor.

53. A composition according to claim 50, a pharmaceutical composition according to claim 51, or a combined preparation according to claim 52, wherein the P-glycoprotein inhibitor is selected from the group consisting of cyclosporine A, ketoconazole, quinidine, ritonavir, verapamil, everolimus, and elacridar.

54. A composition according to claim 50, a pharmaceutical composition according to claim 51, or a combined preparation according to claim 52, wherein the P-glycoprotein inhibitor is quinidine.

55. A pharmaceutical composition, which comprises an inhibitor of EGFR signalling, and an inhibitor of MyD88-dependent TLR/IL-R1 signalling, and a pharmaceutically acceptable carrier, excipient, or diluent, wherein the pharmaceutical composition is suitable for, or adapted for, administration directly to the CNS.

56. A pharmaceutical composition according to claim 55, which comprises one or more electrolytes present in endogenous CSF, wherein the one or more electrolytes is selected from sodium, potassium, calcium, magnesium, phosphorous, and chloride ions.

57. A pharmaceutical composition according to claim 56, which comprises a solution comprising 150 mM sodium ion, 3 mM potassium ion, 1.4 mM calcium ion, 0.8 mM magnesium ion, 1.0 mM phosphorous ion, and 155 mM chloride ion.

58. A pharmaceutical composition according to any of claims 55 to 57, wherein the inhibitor of EGFR signalling, and the inhibitor of MyD88-dependent TLR/IL-R1 signalling is gefitinib.

59. A composition, a pharmaceutical composition, or a combined preparation according to any of claims 50 to 58 for use in the prevention or treatment of a neurodegenerative disease.

60. Use of a composition, a pharmaceutical composition, or a combined preparation according to any of claims 50 to 58 in the manufacture of a medicament for the prevention or treatment of a neurodegenerative disease.

61. Use according to claim 59 or 60, wherein the neurodegenerative disease is a motor neuron disease.

62. Use according to claim 61, wherein the motor neuron disease is amyotrophic lateral sclerosis (ALS).

63. Use according to any of claims 59 to 62, wherein the neurodegenerative disease is a familial neurodegenerative disease.

64. A method according to claim 8, or 20, use according to claim 42, a pharmaceutical composition or a combined preparation according to claim 48, a composition according to claim 50, a pharmaceutical composition according to claim 51, or a combined preparation according to claim 52, wherein the P-glycoprotein inhibitor is elacridar.
